(19) United States
(12) Patent Application Publication
(10) Pub. No.: US 2024/0279195 A1
(43) Pub. Date: Aug. 22, 2024

(54) SOLID STATE FORMS OF ABEXINOSTAT AND PROCESS FOR PREPARATION THEREOF

(71) Applicant: TEVA PHARMACEUTICALS INTERNATIONAL GMBH, Jona (CH)

(72) Inventors: Valentina Travancic, Zagreb (HR); Dijana Skalec Samec, Jastrebarsko (HR); Klara Karadakic, Zegreb (HR); Ivana Landeka, Zegreb (HR)

(21) Appl. No.: 18/567,396

(22) PCT Filed: Jun. 10, 2022
(86) PCT No.: PCT/US2022/032952

§ 371 Date: Dec. 6, 2023

Related U.S. Application Data
(60) Provisional application No. 63/257,634, filed on Oct. 20, 2021.
(60) Provisional application No. 63/228,799, filed on Aug. 3, 2021.
(60) Provisional application No. 63/209,290, filed on Jun. 10, 2021.

(51) Int. Cl. C07D 307/85; A61K 31/343; C07C 57/15; C07C 309/04; C07C 309/05; C07C 309/30

(52) U.S. Cl. CPC C07D 307/85; A61K 31/343; C07C 57/15; C07C 309/04; C07C 309/05; C07C 309/30

(57) ABSTRACT

Solid state forms of Abexinostat salts and of Abexinostat tosylate:p-toluenesulfonic acid monohydrate complex, processes for preparation thereof, pharmaceutical compositions and uses thereof are disclosed.

### FIELD OF THE DISCLOSURE

The present disclosure encompasses solid state forms of Abexinostat, in embodiments crystalline polymorphs or salts of Abexinostat, processes for preparation thereof, and pharmaceutical compositions thereof.

### BACKGROUND OF THE DISCLOSURE

Abexinostat, N-hydroxy-4-[2-(3-dimethylaminomethylbenzofuran)-2-ylcarbonylamino)ethoxy]-benzamide, has the following chemical structure:

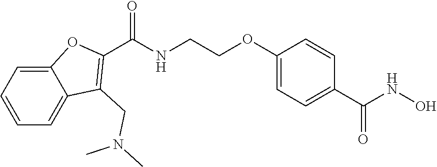 [SMILES: CN(C)Cc1c(C(=O)NCCOc2ccc(C(=O)NO)cc2)oc2ccccc12]

Abexinostat is a hydroxamate-containing histone deacetylase (HDAC) inhibitor, and it is developed for the treatment of cancer.

The compound is described in International Publication No. WO 2004/092115. Salts of Abexinostat are disclosed in International Publication Nos. WO 2010/123507 and WO 2014/135776.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Abexinostat.

### SUMMARY OF THE DISCLOSURE

The present disclosure provides crystalline polymorphs of Abexinostat and Abexinostat salts, processes for preparation thereof, and pharmaceutical compositions thereof. These crystalline polymorphs can be used to prepare other solid state forms of Abexinostat, Abexinostat salts and their solid state forms.

The present disclosure also provides uses of the said solid state forms of Abexinostat or Abexinostat salts in the preparation of other solid state forms of Abexinostat or salts thereof.

The present disclosure provides crystalline polymorphs of Abexinostat or Abexinostat salts for use in medicine, including for the treatment of cancer.

The present disclosure also encompasses the use of crystalline polymorphs of Abexinostat or Abexinostat salts of the present disclosure for the preparation of pharmaceutical compositions and/or formulations.

In another aspect, the present disclosure provides pharmaceutical compositions comprising crystalline polymorphs of Abexinostat or Abexinostat salts according to the present disclosure.

The present disclosure includes processes for preparing the above mentioned pharmaceutical compositions. The processes include combining any one or a combination of the crystalline polymorphs of Abexinostat or Abexinostat salt with at least one pharmaceutically acceptable excipient.

The crystalline polymorph of Abexinostat or Abexinostat salt as defined herein and the pharmaceutical compositions or formulations of the crystalline polymorph of Abexinostat or Abexinostat salt may be used as medicaments, such as for the treatment of cancer.

The present disclosure also provides methods of treating cancer, by administering a therapeutically effective amount of any one or a combination of the crystalline polymorphs of Abexinostat or Abexinostat salt of the present disclosure, or at least one of the above pharmaceutical compositions, to a subject suffering from cancer, or otherwise in need of the treatment.

The present disclosure also provides uses of crystalline polymorphs of Abexinostat or Abexinostat salt of the present disclosure, or at least one of the above pharmaceutical compositions, for the manufacture of medicaments for treating e.g., cancer.

### BRIEF DESCRIPTION OF THE DRAWINGS

FIG. 1 shows a characteristic X-ray powder diffraction pattern (XRPD) of Abexinostat Form A.

FIG. 2 shows a characteristic XRPD of Abexinostat HCl, Form H1.

FIG. 3 shows a characteristic XRPD of Abexinostat HCl, Form H2.

FIG. 4 shows a characteristic XRPD of Abexinostat fumarate, Form F1.

FIG. 5 shows a characteristic XRPD of Abexinostat maleate, Form M1.

FIG. 6 shows a characteristic XRPD of Abexinostat fumarate, Form F2.

FIG. 7 shows a characteristic XRPD of Abexinostat mesylate, Form MS1.

FIG. 8 shows a characteristic XRPD of Abexinostat edisylate, Form E1.

FIG. 9 shows a characteristic XRPD of Abexinostat tosylate, Form T1.

FIG. 10 shows a characteristic XRPD of Abexinostat tosylate, Form T2.

FIG. 11 shows a characteristic XRPD of Abexinostat tosylate, Form T3.

FIG. 12 shows a characteristic XRPD of Abexinostat fumarate, Form F3.

FIG. 13 shows a characteristic XRPD of Abexinostat edisylate, Form E2.

FIG. 14 shows a characteristic XRPD of Abexinostat esylate, Form ES1.

FIG. 15 shows a characteristic XRPD of Abexinostat esylate, Form ES5.

FIG. 16 shows a characteristic XRPD of Abexinostat esylate, Form ES6.

FIG. 17 shows a characteristic XRPD of Abexinostat edisylate, Form E6.

FIG. 18 shows a characteristic XRPD of Abexinostat edisylate, Form E7.

FIG. 19 shows a characteristic XRPD of Abexinostat tosylate, Form T1.

FIG. 20 shows a characteristic DSC thermogram of Abexinostat maleate Form M1.

FIG. 21 shows a characteristic DSC thermogram of Abexinostat fumarate Form F3.

FIG. 22 shows a characteristic DSC thermogram of Abexinostat tosylate Form T1.

FIG. 23 shows a characteristic solid state ¹³C-NMR spectrum of Abexinostat tosylate, Form T1 (FIG. 23a: 200-0 ppm; FIG. 23b: 200-100 ppm; FIG. 23c: 100-0 ppm).

### DETAILED DESCRIPTION OF THE DISCLOSURE

The present disclosure encompasses solid state forms of Abexinostat and Abexinostat salts, including crystalline polymorphs of Abexinostat or Abexinostat salts, processes for preparation thereof, and pharmaceutical compositions thereof.

Solid state properties of Abexinostat or Abexinostat salts and crystalline polymorphs thereof can be influenced by controlling the conditions under which Abexinostat or Abexinostat salts and crystalline polymorphs thereof are obtained in solid form.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Abexinostat described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Abexinostat or salt thereof. In some embodiments of the disclosure, the described crystalline polymorph of Abexinostat or Abexinostat salt may contain from about 1% to about 20% (w/w), from about 5% to about 20% (w/w), or from about 5% to about 10% (w/w) of one or more other crystalline polymorph of the same Abexinostat or Abexinostat salt.

Depending on which other crystalline polymorphs a comparison is made, the crystalline polymorphs of Abexinostat or Abexinostat salts of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Abexinostat referred to herein as being characterized by graphical data “as depicted in” or “as substantially depicted in” a Figure will thus be understood to include any crystal forms of Abexinostat characterized with the graphical data having such small variations, as are well known to the skilled person, in comparison with the Figure.

As used herein, and unless stated otherwise, the term “anhydrous” in relation to crystalline forms of Abexinostat, relates to a crystalline form of Abexinostat which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

The term “complex” as used herein and unless indicated otherwise, refers to a crystal form that incorporates an ionic liquid in the crystal structure. The ionic liquid may be present in either ionic or non-ionic form.

“Co-Crystal” as used herein is defined as a crystalline material including two or more molecules in the same crystalline lattice and associated by non-ionic and non-covalent bonds. In some embodiments, the co-crystal includes two molecules which are in natural state. In an embodiments the molar ratio between the active pharmaceutical ingredient Abexinostat tosylate and the coformer p-toluenesulfonic acid is between 1:1.5 and 1.5:1, preferably between 1:1.25 and 1.25:1, in other embodiments about 1:1.

As used herein, the term “isolated” in reference to crystalline polymorph of Abexinostat of the present disclosure corresponds to a crystalline polymorph of Abexinostat that is physically separated from the reaction mixture in which it is formed.

As used herein, unless stated otherwise, the XRPD measurements are taken using copper Kα radiation wavelength 1.5418 Å. XRPD peaks reported herein are measured using CuK α radiation, λ=1.5418 Å, typically at a temperature of 25±3° C.

A thing, e.g., a reaction mixture, may be characterized herein as being at, or allowed to come to “room temperature” or “ambient temperature,” often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

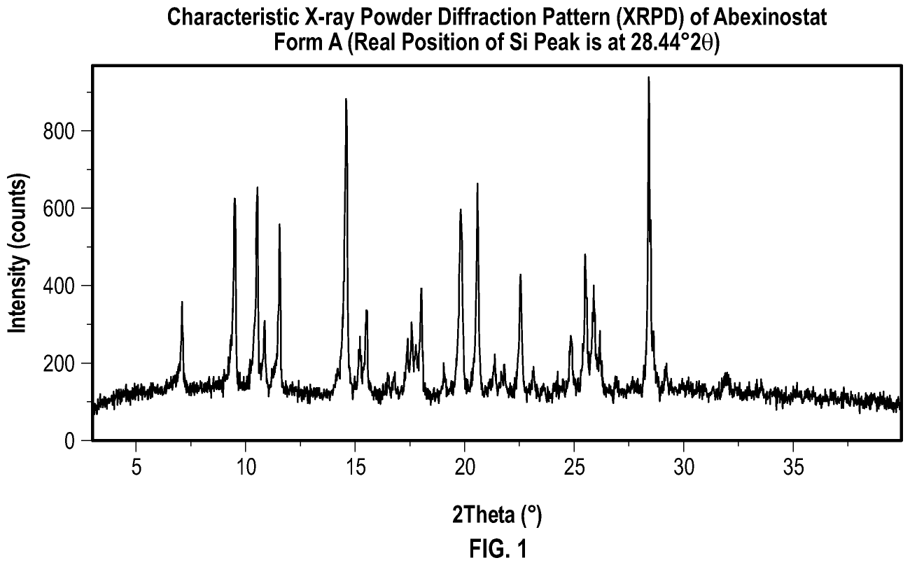

The present disclosure includes a crystalline polymorph of Abexinostat, designated Form A. The crystalline Form A of Abexinostat may be characterized by data selected from one or more of the following: an X-ray powder diffraction pattern substantially as depicted in FIG. 1; an X-ray powder diffraction pattern having peaks at 9.5, 14.6, 15.6, 18.1, and 20.6 degrees 2-theta±0.2 degrees 2-theta; and combinations of these data.

Crystalline Form A of Abexinostat may be further characterized by an X-ray powder diffraction pattern having peaks at 9.5, 14.6, 15.6, 18.1, and 20.6 degrees 2-theta±0.2 degrees 2-theta, and also having any one, two, three, four or five additional peaks selected from 7.1, 10.5, 17.4, 19.1 and 25.5 degrees 2-theta.

In one embodiment of the present disclosure, crystalline Form A of Abexinostat is isolated.

Crystalline Form A of Abexinostat may be anhydrous form.

Crystalline Form A of Abexinostat may be characterized by each of the above characteristics alone/or by all possible combinations, e.g., an XRPD pattern having peaks at 9.5, 14.6, 15.6, 18.1, and 20.6 degrees 2-theta±0.2 degrees 2-theta; an XRPD pattern as depicted in FIG. 1, and combinations thereof.

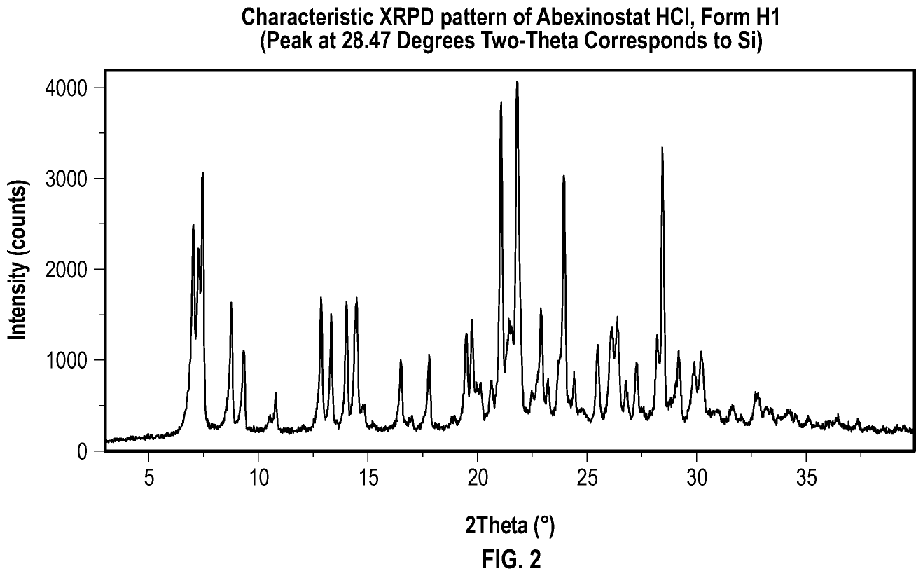

The present disclosure further includes a crystalline polymorph of Abexinostat HCl, designated Form H1. The crystalline Form H1 of Abexinostat HCl may be characterized by data selected from one or more of the following: an X-ray powder diffraction pattern substantially as depicted in FIG. 2; an X-ray powder diffraction pattern having peaks at 7.4, 8.8, 21.1, 21.8 and 24.0 degrees 2-theta±0.2 degrees 2-theta; and combinations of these data.

Crystalline Form H1 of Abexinostat HCl may be further characterized by an X-ray powder diffraction pattern having peaks at 7.4, 8.8, 21.1, 21.8 and 24.0 degrees 2-theta±0.2 degrees 2-theta, and also having any one, two, three, four or five additional peaks selected from 12.9, 13.3, 14.0, 14.5 and 22.9 degrees 2-theta±0.2 degrees 2-theta.

In one embodiment of the present disclosure, crystalline Form H1 of Abexinostat HCl is isolated.

Crystalline Form H1 of Abexinostat HCl may be a hydrated form. Crystalline Form H1 of Abexinostat HCl may be a dihydrate.

Crystalline Form H1 of Abexinostat HCl may be characterized by each of the above characteristics alone/or by all possible combinations, e.g., an XRPD pattern having peaks at 7.4, 8.8, 21.1, 21.8 and 24.0 degrees 2-theta±0.2 degrees 2-theta; an XRPD pattern as depicted in FIG. 2, and combinations thereof.

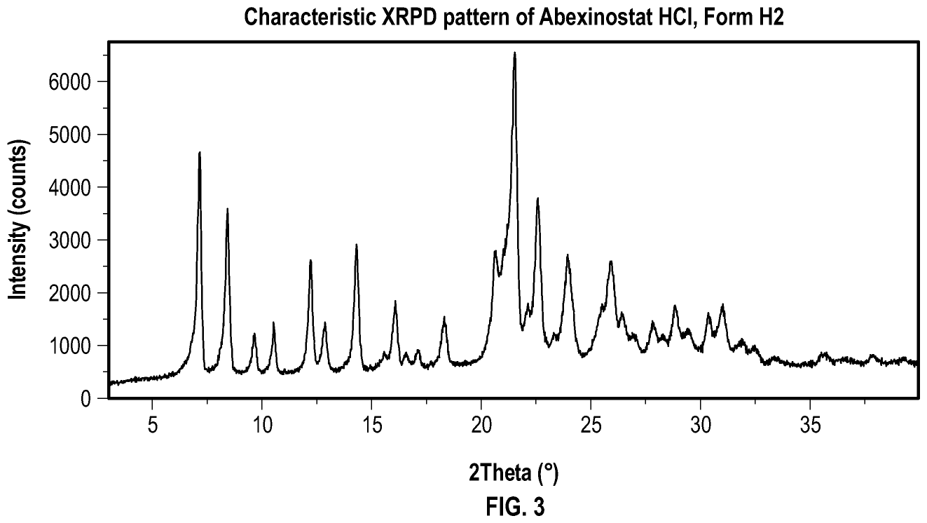

The present disclosure further includes a crystalline polymorph of Abexinostat HCl, designated Form H2. The crystalline Form H2 of Abexinostat HCl may be characterized by data selected from one or more of the following: an X-ray powder diffraction pattern substantially as depicted in FIG. 3; an X-ray powder diffraction pattern having peaks at 8.4, 12.2, 16.1 and 18.3 degrees 2-theta±0.2 degrees 2-theta; and combinations of these data.

Crystalline Form H2 of Abexinostat HCl may be further characterized by an X-ray powder diffraction pattern having peaks at 8.4, 12.2, 16.1 and 18.3 degrees 2-theta±0.2 degrees 2-theta, and also having any one, two, or three additional peaks selected from 27.9, 30.4, and 31.1 degrees 2-theta±0.2 degrees 2-theta.

In one embodiment of the present disclosure, crystalline Form H2 of Abexinostat HCl is isolated.

Crystalline Form H2 of Abexinostat HCl may be an anhydrous form.

Crystalline Form H2 of Abexinostat HCl may be characterized by each of the above characteristics alone/or by all possible combinations, e.g., an XRPD pattern having peaks at 8.4, 12.2, 16.1 and 18.3 degrees 2-theta±0.2 degrees 2-theta; an XRPD pattern as depicted in FIG. 3, and combinations thereof.

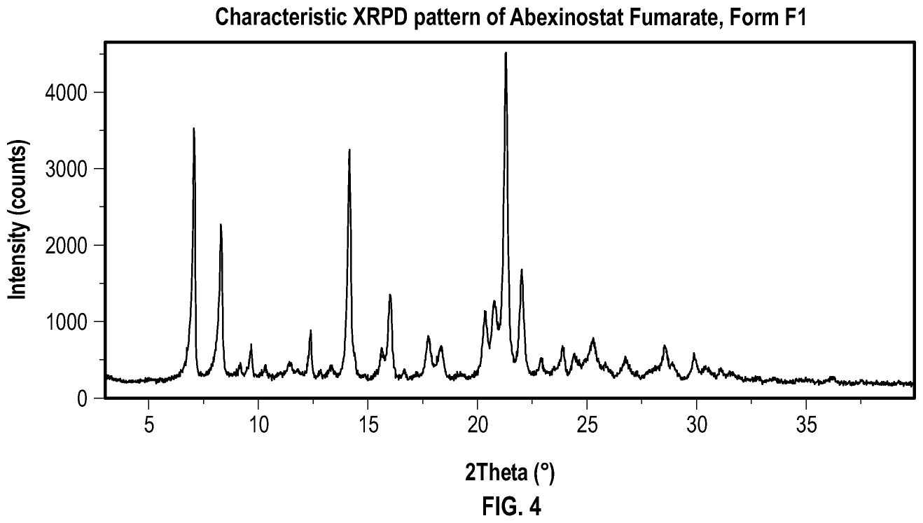

The present disclosure further includes a crystalline polymorph of Abexinostat fumarate, designated Form F1. The crystalline Form F1 of Abexinostat fumarate may be characterized by data selected from one or more of the following: an X-ray powder diffraction pattern substantially as depicted in FIG. 4; an X-ray powder diffraction pattern having peaks at 8.3, 14.2, 16.1, and 22.0 degrees 2-theta±0.2 degrees 2-theta; and combinations of these data.

Crystalline Form F1 of Abexinostat fumarate may be further characterized by an X-ray powder diffraction pattern having peaks at 8.3, 14.2, 16.1, and 22.0 degrees 2-theta±0.2 degrees 2-theta, and also having any one, two, three or four additional peaks selected from 12.4, 20.3, 25.3, and 28.6 degrees 2-theta±0.2 degrees 2-theta.

In one embodiment of the present disclosure, crystalline Form F1 of Abexinostat fumarate is isolated.

Crystalline Form F1 of Abexinostat fumarate may be characterized by each of the above characteristics alone/or by all possible combinations, e.g., an XRPD pattern having peaks at 8.3, 14.2, 16.1, and 22.0 degrees 2-theta±0.2 degrees 2-theta; an XRPD pattern as depicted in FIG. 4, and combinations thereof.

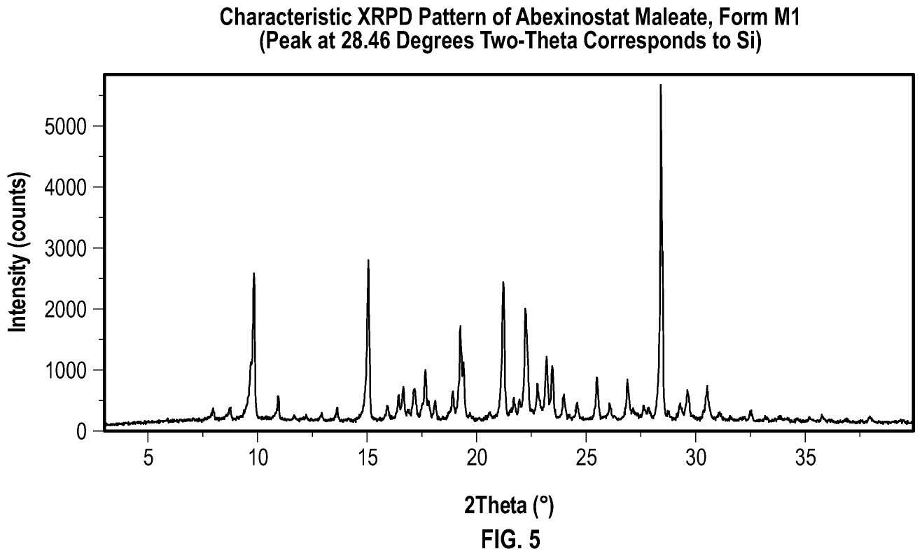

The present disclosure further includes a crystalline polymorph of Abexinostat maleate, designated Form M1. The crystalline Form M1 of Abexinostat maleate may be characterized by data selected from one or more of the following: an X-ray powder diffraction pattern substantially as depicted in FIG. 5; an X-ray powder diffraction pattern having peaks at 9.8, 15.1, 22.3 and 23.5 degrees 2-theta±0.2 degrees 2-theta; and combinations of these data.

Crystalline Form M1 of Abexinostat maleate may be further characterized by an X-ray powder diffraction pattern having peaks at 9.8, 15.1, 22.3 and 23.5 degrees 2-theta±0.2 degrees 2-theta, and also having any one, two, three, or four additional peaks selected from 9.7, 19.3, 21.2 and 22.8 degrees 2-theta±0.2 degrees 2-theta.

In one embodiment of the present disclosure, crystalline Form M1 of Abexinostat maleate is isolated.

Crystalline Form M1 of Abexinostat maleate may be anhydrous. The TGA thermogram of crystalline Form M1 of Abexinostat maleate shows a weight loss of less than 1% up to 144° C., preferably less than 0.7%, most preferably around 0.5%.

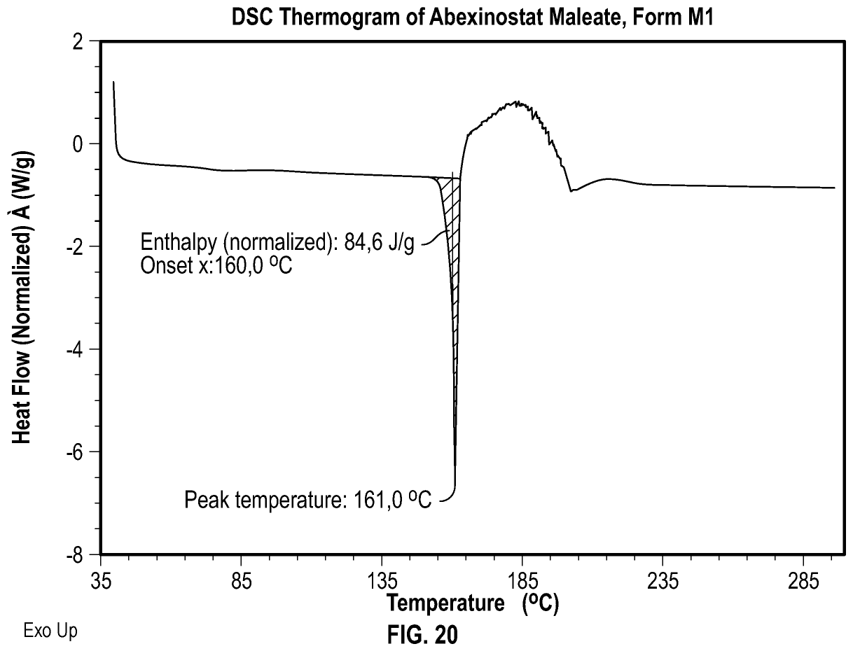

Crystalline Form M1 of Abexinostat maleate may alternatively or additionally be characterized by a DSC thermogram as depicted in FIG. 20. Crystalline Form M1 of Abexinostat maleate shows one endothermic peak with an onset at 160° C.±5° C. and a melting point at around 161° C.

Crystalline Form M1 of Abexinostat maleate is stable upon heating, pressure, grinding and upon exposure to humidity.

Crystalline Form M1 of Abexinostat maleate may be characterized by each of the above characteristics alone/or by all possible combinations, e.g., an XRPD pattern having peaks at 9.8, 15.1, 22.3 and 23.5 degrees 2-theta±0.2 degrees 2-theta; an XRPD pattern as depicted in FIG. 5, and combinations thereof.

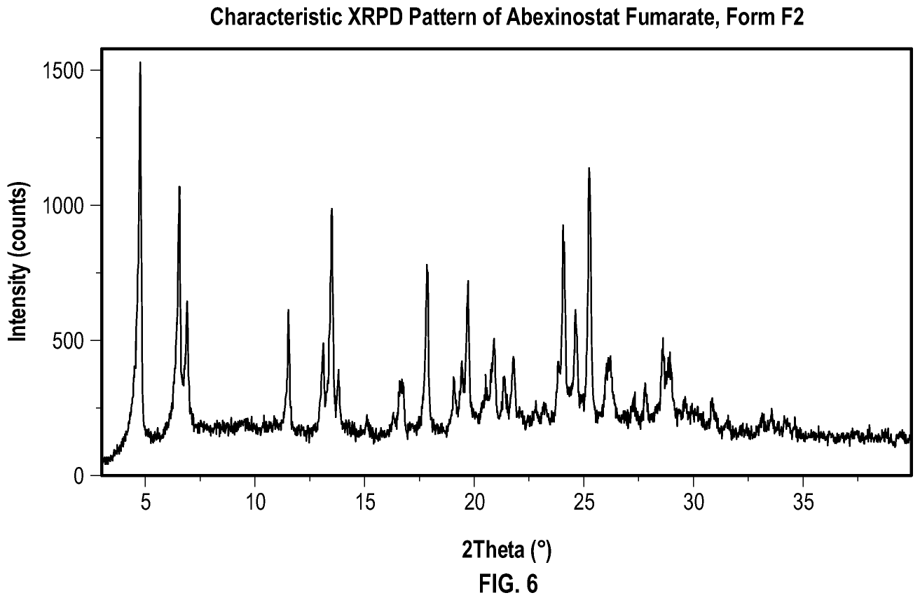

The present disclosure further includes a crystalline polymorph of Abexinostat fumarate, designated Form F2. The crystalline Form F2 of Abexinostat fumarate may be characterized by data selected from one or more of the following: an X-ray powder diffraction pattern substantially as depicted in FIG. 6; an X-ray powder diffraction pattern having peaks at 4.8, 6.6, 13.5 and 24.1 degrees 2-theta±0.2 degrees 2-theta; and combinations of these data.

Crystalline Form F2 of Abexinostat fumarate may be further characterized by an X-ray powder diffraction pattern having peaks at 4.8, 6.6, 13.5 and 24.1 degrees 2-theta±0.2 degrees 2-theta, and also having any one, two, three, or four additional peaks selected from 13.1, 13.8, 19.5 and 24.7 degrees 2-theta±0.2 degrees 2-theta.

In one embodiment of the present disclosure, crystalline Form F2 of Abexinostat fumarate is isolated.

Crystalline Form F2 of Abexinostat fumarate may be a hydrated form.

Crystalline Form F2 of Abexinostat fumarate may be characterized by each of the above characteristics alone/or by all possible combinations, e.g., an XRPD pattern having peaks at 4.8, 6.6, 13.5 and 24.1 degrees 2-theta±0.2 degrees 2-theta; an XRPD pattern as depicted in FIG. 6, and combinations thereof.

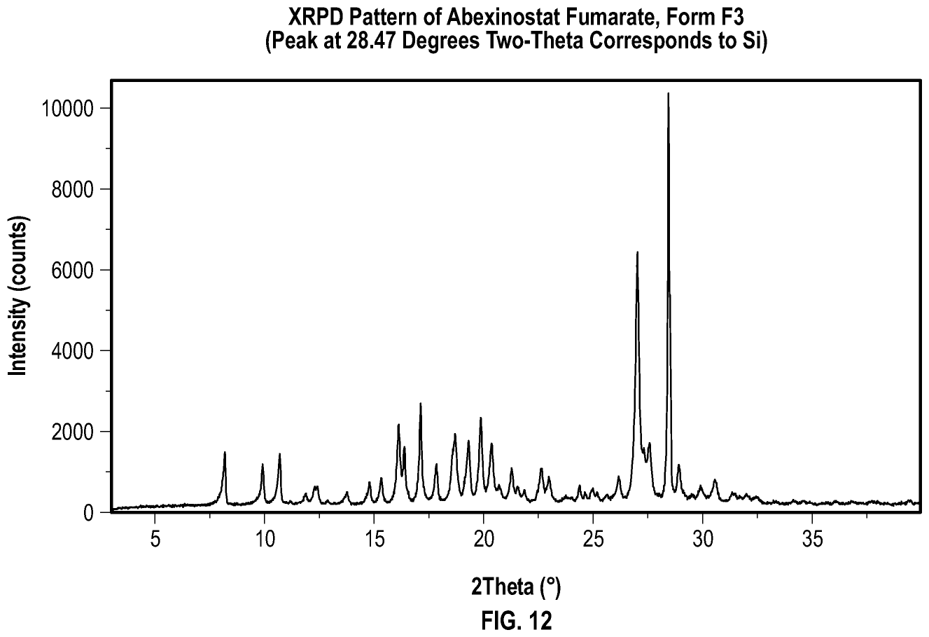

The present disclosure further includes a crystalline polymorph of Abexinostat fumarate, designated Form F3. The crystalline Form F1 of Abexinostat fumarate may be characterized by data selected from one or more of the following: an X-ray powder diffraction pattern substantially as depicted in FIG. 12; an X-ray powder diffraction pattern having peaks at 9.9, 10.7, 17.1, 18.7 and 27.0 degrees 2-theta±0.2 degrees 2-theta; and combinations of these data.

Crystalline Form F3 of Abexinostat fumarate may be further characterized by an X-ray powder diffraction pattern having peaks at 9.9, 10.7, 17.1, 18.7 and 27.0 degrees 2-theta±0.2 degrees 2-theta, and also having any one, two, three, or four additional peaks selected from 15.3, 16.1, 16.4 and 26.2 degrees 2-theta±0.2 degrees 2-theta.

In one embodiment of the present disclosure, crystalline Form F3 of Abexinostat fumarate is isolated.

Crystalline Form F3 of Abexinostat fumarate may be an anhydrous form. The TGA thermogram of crystalline Form F3 of Abexinostat fumarate shows a weight loss of less than 1% up to 144° C., preferably less than 0.7%, most preferably less than 0.5%.

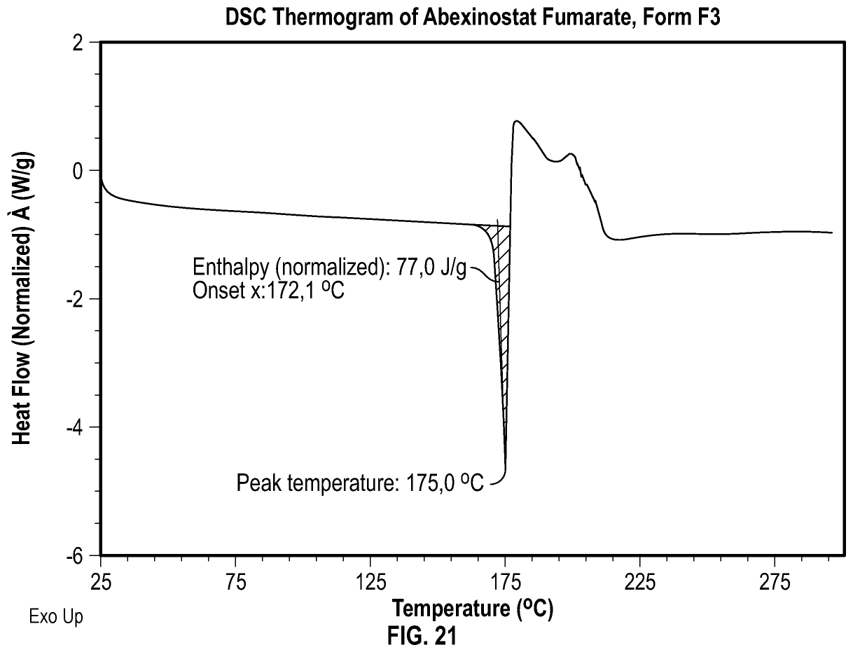

Crystalline Form F3 of Abexinostat fumarate may alternatively or additionally be characterized by a DSC thermogram as depicted in FIG. 21. Crystalline Form F3 of Abexinostat fumarate shows one endothermic peak with an onset at 171° C.±3° C. and a melting point at around 176° C.

Crystalline Form F3 of Abexinostat fumarate is stable upon heating, pressure, grinding and upon exposure to humidity.

Crystalline Form F3 of Abexinostat fumarate may be characterized by each of the above characteristics alone/or by all possible combinations, e.g., an XRPD pattern having peaks at 9.9, 10.7, 17.1, 18.7 and 27.0 degrees 2-theta±0.2 degrees 2-theta; an XRPD pattern as depicted in FIG. 12, and combinations thereof.

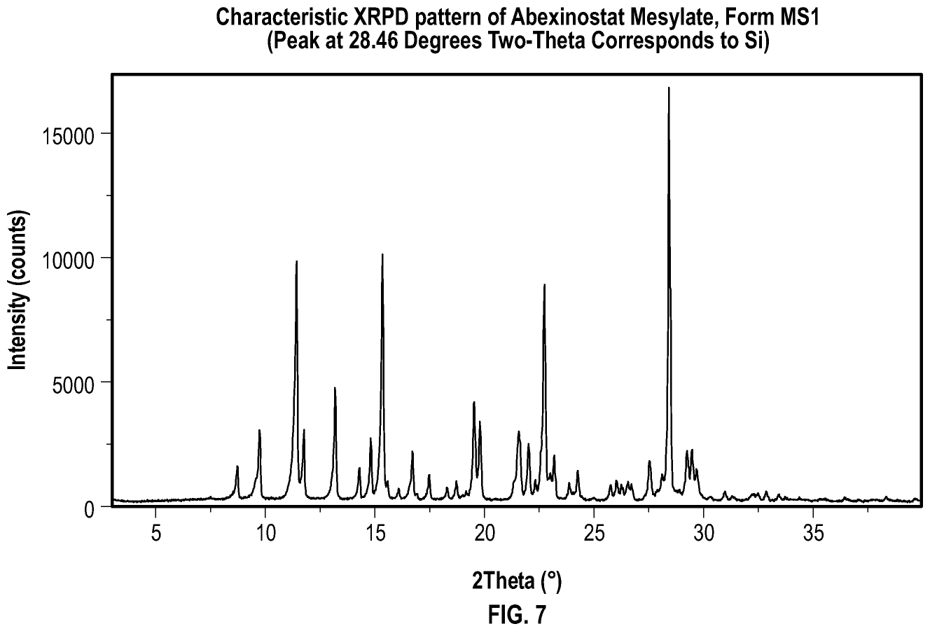

The present disclosure further includes a crystalline polymorph of Abexinostat mesylate, designated Form MS1. The crystalline Form MS1 of Abexinostat mesylate may be characterized by data selected from one or more of the following: an X-ray powder diffraction pattern substantially as depicted in FIG. 7; an X-ray powder diffraction pattern having peaks at 11.4, 13.2, 15.4, 19.6 and 22.8 degrees 2-theta±0.2 degrees 2-theta; and combinations of these data.

Crystalline Form MS1 of Abexinostat mesylate may be further characterized by an X-ray powder diffraction pattern having peaks at 11.4, 13.2, 15.4, 19.6 and 22.8 degrees 2-theta 0.2 degrees 2-theta, and also having any one, two, three, four, or five additional peaks selected from 9.8, 11.8, 16.7, 21.7 and 28.5 degrees 2-theta±0.2 degrees 2-theta.

In one embodiment of the present disclosure, crystalline Form MS1 of Abexinostat mesylate is isolated.

Crystalline Form MS1 of Abexinostat mesylate may be an anhydrous form.

Crystalline Form MS1 of Abexinostat mesylate may be characterized by each of the above characteristics alone/or by all possible combinations, e.g., an XRPD pattern having peaks at 11.4, 13.2, 15.4, 19.6 and 22.8 degrees 2-theta±0.2 degrees 2-theta; an XRPD pattern as depicted in FIG. 7, and combinations thereof.

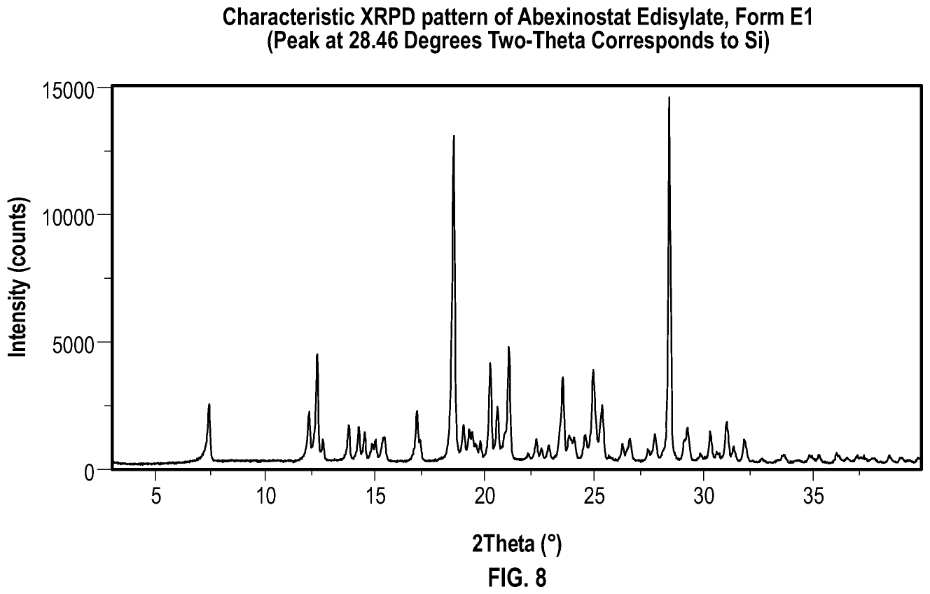

The present disclosure further includes a crystalline polymorph of Abexinostat edisylate, designated Form E1. The crystalline Form E1 of Abexinostat edisylate may be characterized by data selected from one or more of the following: an X-ray powder diffraction pattern substantially as depicted in FIG. 8; an X-ray powder diffraction pattern having peaks at 7.4, 12.4, 13.8, 18.6 and 20.3 degrees 2-theta±0.2 degrees 2-theta; and combinations of these data.

Crystalline Form E1 of Abexinostat edisylate may be further characterized by an X-ray powder diffraction pattern having peaks at 7.4, 12.4, 13.8, 18.6 and 20.3 degrees 2-theta±0.2 degrees 2-theta, and also having any one, two, three, four or five additional peaks selected from 12.0, 19.4, 21.1, 22.9 and 23.6 degrees 2-theta±0.2 degrees 2-theta.

In one embodiment of the present disclosure, crystalline Form E1 of Abexinostat edisylate is isolated.

Crystalline Form E1 of Abexinostat edisylate may be an anhydrous form. Crystalline Form E1 of Abexinostat edisylate may be a hydrated form.

Crystalline Form E1 of Abexinostat edisylate may be characterized by each of the above characteristics alone/or by all possible combinations, e.g., an XRPD pattern having peaks at 7.4, 12.4, 13.8, 18.6 and 20.3 degrees 2-theta±0.2 degrees 2-theta; an XRPD pattern as depicted in FIG. 8, and combinations thereof.

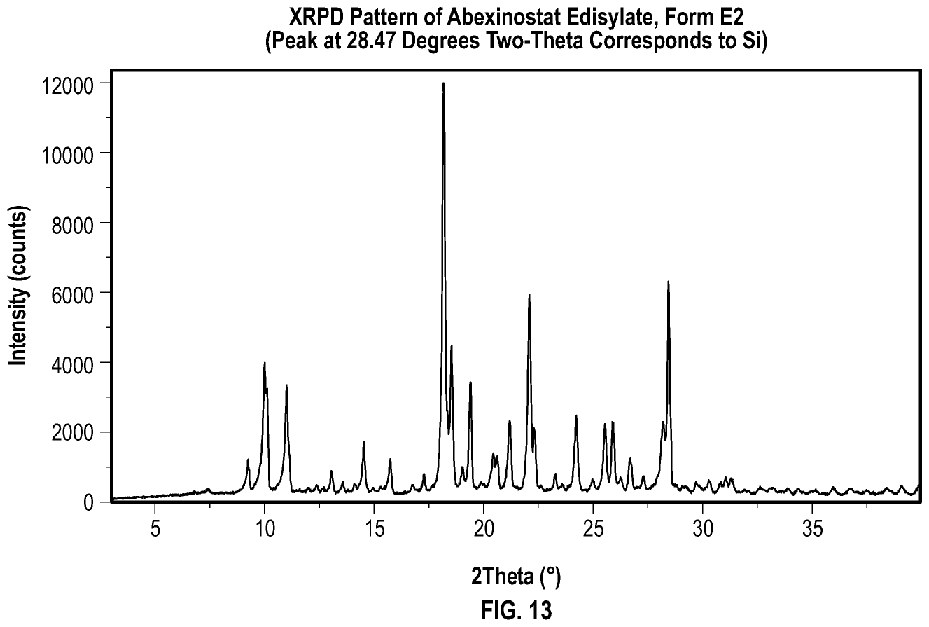

The present disclosure further includes a crystalline polymorph of Abexinostat edisylate, designated Form E2. The crystalline Form E2 of Abexinostat edisylate may be characterized by data selected from one or more of the following: an X-ray powder diffraction pattern substantially as depicted in FIG. 13; an X-ray powder diffraction pattern having peaks at 10.0, 11.0, 13.1, 22.1 and 24.2 degrees 2-theta±0.2 degrees 2-theta; and combinations of these data.

Crystalline Form E2 of Abexinostat edisylate may be further characterized by an X-ray powder diffraction pattern having peaks at 10.0, 11.0, 13.1, 22.1 and 24.2 degrees 2-theta±0.2 degrees 2-theta, and also having any one, two, three, or four additional peaks selected from 9.2, 15.7, 17.3 and 18.2 degrees 2-theta±0.2 degrees 2-theta.

In one embodiment of the present disclosure, crystalline Form E2 of Abexinostat edisylate is isolated.

Crystalline Form E2 of Abexinostat edisylate may be an anhydrous form. Crystalline Form E2 of Abexinostat edisylate may be a hydrated form.

Crystalline Form E2 of Abexinostat edisylate may be characterized by each of the above characteristics alone/or by all possible combinations, e.g., an XRPD pattern having peaks at 10.0, 11.0, 13.1, 22.1 and 24.2 degrees 2-theta±0.2 degrees 2-theta; an XRPD pattern as depicted in FIG. 13, and combinations thereof.

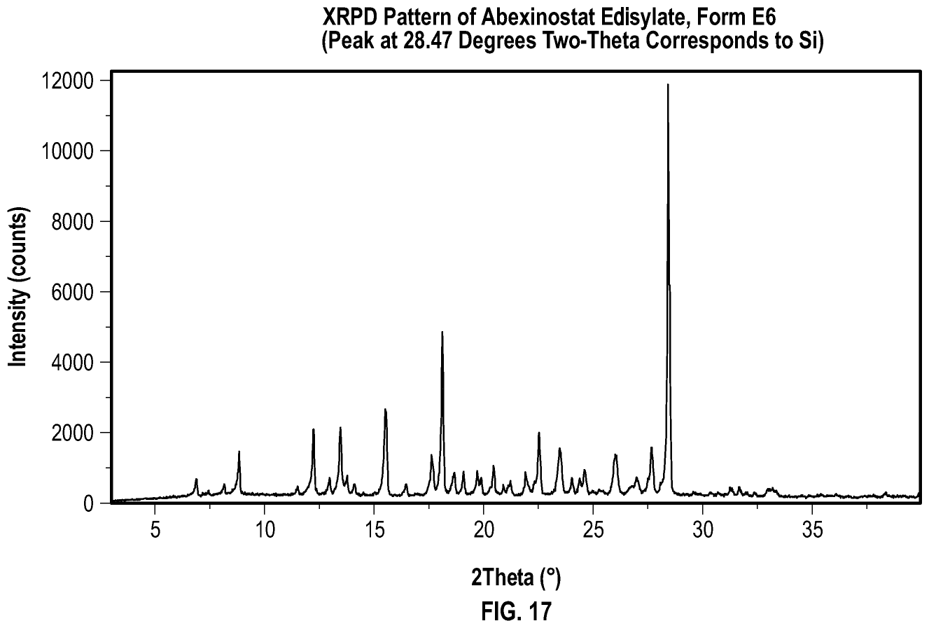

The present disclosure further includes a crystalline polymorph of Abexinostat edisylate, designated Form E6. The crystalline Form E6 of Abexinostat edisylate may be characterized by data selected from one or more of the following: an X-ray powder diffraction pattern substantially as depicted in FIG. 17; an X-ray powder diffraction pattern having peaks at 6.9, 8.2, 8.8, 13.5 and 22.5 degrees 2-theta±0.2 degrees 2-theta; and combinations of these data.

Crystalline Form E6 of Abexinostat edisylate may be further characterized by an X-ray powder diffraction pattern having peaks at 6.9, 8.2, 8.8, 13.5 and 22.5 degrees 2-theta±0.2 degrees 2-theta, and also having any one, two, three, or four additional peaks selected from 11.5, 13.0, 15.5, 16.5 and 17.6 degrees 2-theta±0.2 degrees 2-theta.

In one embodiment of the present disclosure, crystalline Form E6 of Abexinostat edisylate is isolated.

Crystalline Form E6 of Abexinostat edisylate may be an anhydrous form. Crystalline Form E6 of Abexinostat edisylate may be a hydrated form.

Crystalline Form E6 of Abexinostat edisylate may be characterized by each of the above characteristics alone/or by all possible combinations, e.g., an XRPD pattern having peaks at 6.9, 8.2, 8.8, 13.5 and 22.5 degrees 2-theta±0.2 degrees 2-theta; an XRPD pattern as depicted in FIG. 17, and combinations thereof.

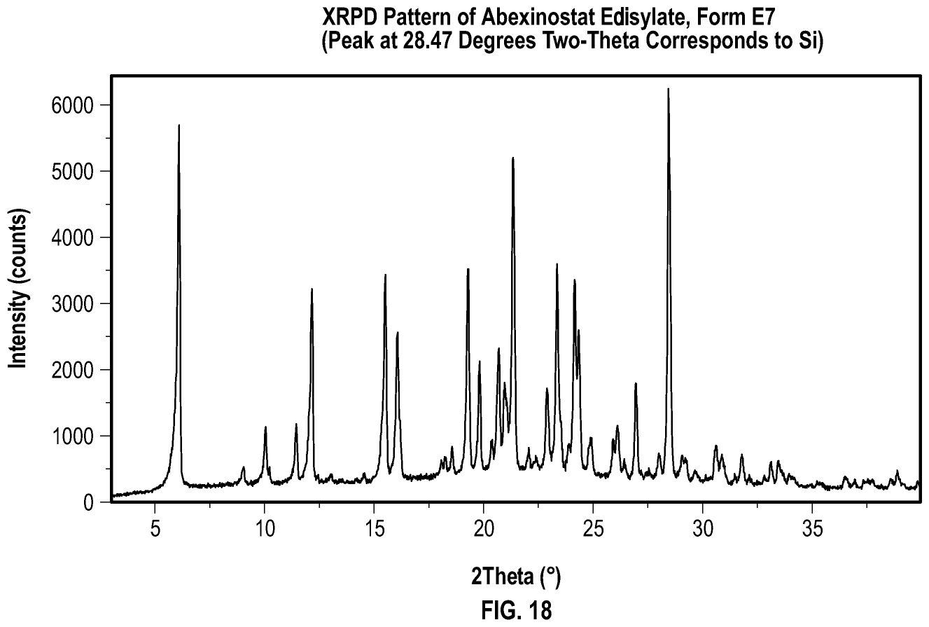

The present disclosure further includes a crystalline polymorph of Abexinostat edisylate, designated Form E7. The crystalline Form E7 of Abexinostat edisylate may be characterized by data selected from one or more of the following: an X-ray powder diffraction pattern substantially as depicted in FIG. 18; an X-ray powder diffraction pattern having peaks at 6.1, 12.2, 16.1 and 21.3 degrees 2-theta±0.2 degrees 2-theta; and combinations of these data.

Crystalline Form E7 of Abexinostat edisylate may be further characterized by an X-ray powder diffraction pattern having peaks at 6.1, 12.2, 16.1 and 21.3 degrees 2-theta±0.2 degrees 2-theta, and also having any one, two, three, or four additional peaks selected from 9.0, 11.4, 23.4 and 27.0 degrees 2-theta±0.2 degrees 2-theta.

In one embodiment of the present disclosure, crystalline Form E7 of Abexinostat edisylate is isolated.

Crystalline Form E7 of Abexinostat edisylate may be an anhydrous form.

Crystalline Form E7 of Abexinostat edisylate may be characterized by each of the above characteristics alone/or by all possible combinations, e.g., an XRPD pattern having peaks at 6.1, 12.2, 16.1 and 21.3 degrees 2-theta±0.2 degrees 2-theta; an XRPD pattern as depicted in FIG. 18, and combinations thereof.

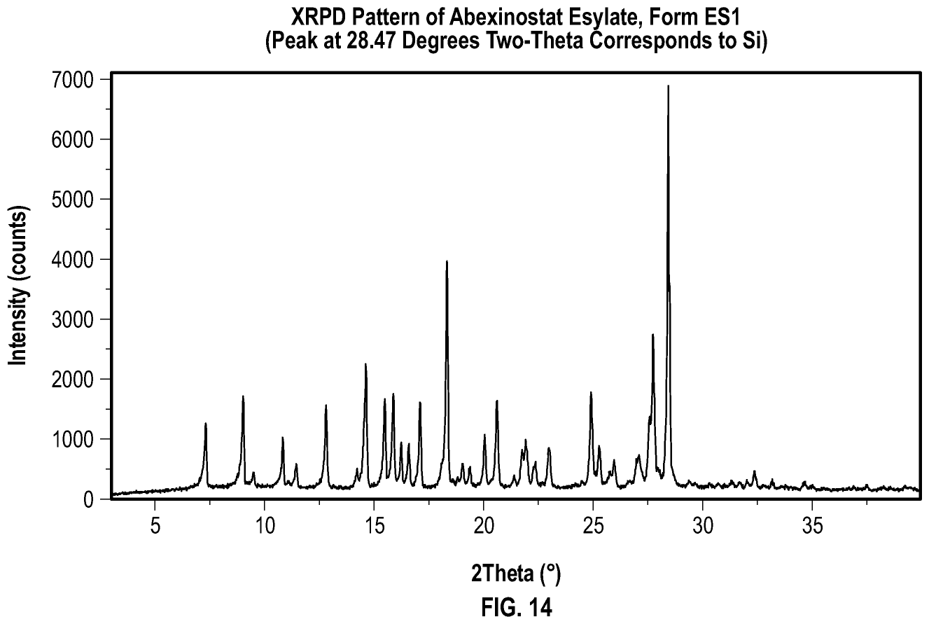

The present disclosure further includes a crystalline polymorph of Abexinostat esylate, designated Form ES1. The crystalline Form ES1 of Abexinostat esylate may be characterized by data selected from one or more of the following: an X-ray powder diffraction pattern substantially as depicted in FIG. 14; an X-ray powder diffraction pattern having peaks at 9.0, 15.9, 20.1 and 23.0 degrees 2-theta±0.2 degrees 2-theta; and combinations of these data.

Crystalline Form ES1 of Abexinostat esylate may be further characterized by an X-ray powder diffraction pattern having peaks at 9.0, 15.9, 20.1 and 23.0 degrees 2-theta±0.2 degrees 2-theta, and also having any one, two, three, or four additional peaks selected from 7.3, 21.9, 24.9 and 27.8 degrees 2-theta±0.2 degrees 2-theta.

In one embodiment of the present disclosure, crystalline Form ES1 of Abexinostat esylate is isolated.

Crystalline Form ES1 of Abexinostat esylate may be an anhydrous form. Crystalline Form ES1 of Abexinostat esylate may be a hydrated form.

Crystalline Form ES1 of Abexinostat esylate may be characterized by each of the above characteristics alone/or by all possible combinations, e.g., an XRPD pattern having peaks at 9.0, 15.9, 20.1 and 23.0 degrees 2-theta±0.2 degrees 2-theta; an XRPD pattern as depicted in FIG. 14, and combinations thereof.

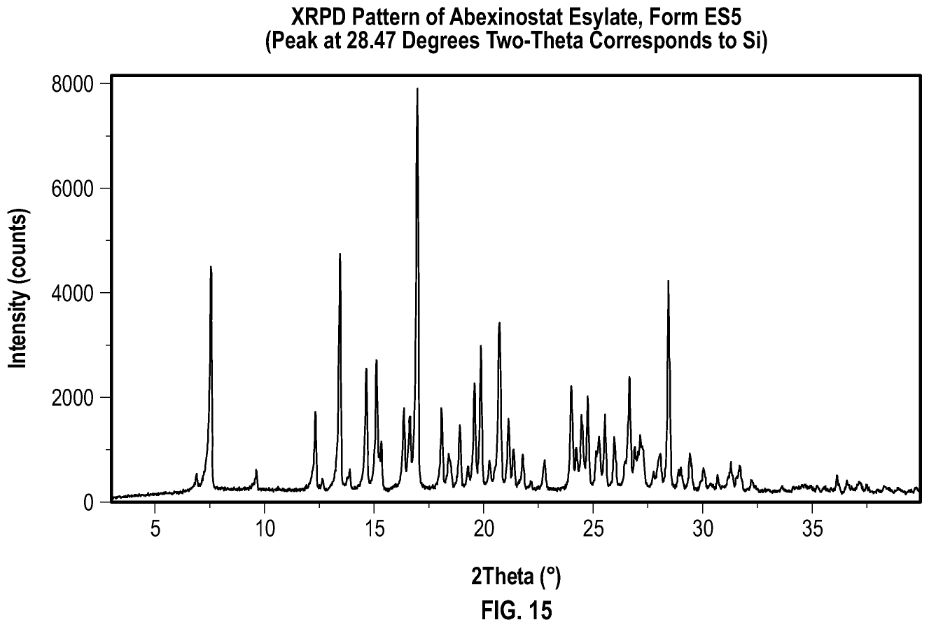

The present disclosure further includes a crystalline polymorph of Abexinostat esylate, designated Form ES5. The crystalline Form ES5 of Abexinostat esylate may be characterized by data selected from one or more of the following: an X-ray powder diffraction pattern substantially as depicted in FIG. 15; an X-ray powder diffraction pattern having peaks at 7.6, 12.3, 13.5 and 18.9 degrees 2-theta±0.2 degrees 2-theta; and combinations of these data.

Crystalline Form ES5 of Abexinostat esylate may be further characterized by an X-ray powder diffraction pattern having peaks at 7.6, 12.3, 13.5 and 18.9 degrees 2-theta±0.2 degrees 2-theta, and also having any one, two, three, or four additional peaks selected from 16.4, 20.7, 24.0 and 24.7 degrees 2-theta±0.2 degrees 2-theta.

In one embodiment of the present disclosure, crystalline Form ES5 of Abexinostat esylate is isolated.

Crystalline Form ES5 of Abexinostat esylate may be an anhydrous form. Crystalline Form ES5 of Abexinostat esylate may be a hydrated form.

Crystalline Form ES1 of Abexinostat esylate may be characterized by each of the above characteristics alone/or by all possible combinations, e.g., an XRPD pattern having peaks at 7.6, 12.3, 13.5 and 18.9 degrees 2-theta±0.2 degrees 2-theta; an XRPD pattern as depicted in FIG. 15, and combinations thereof.

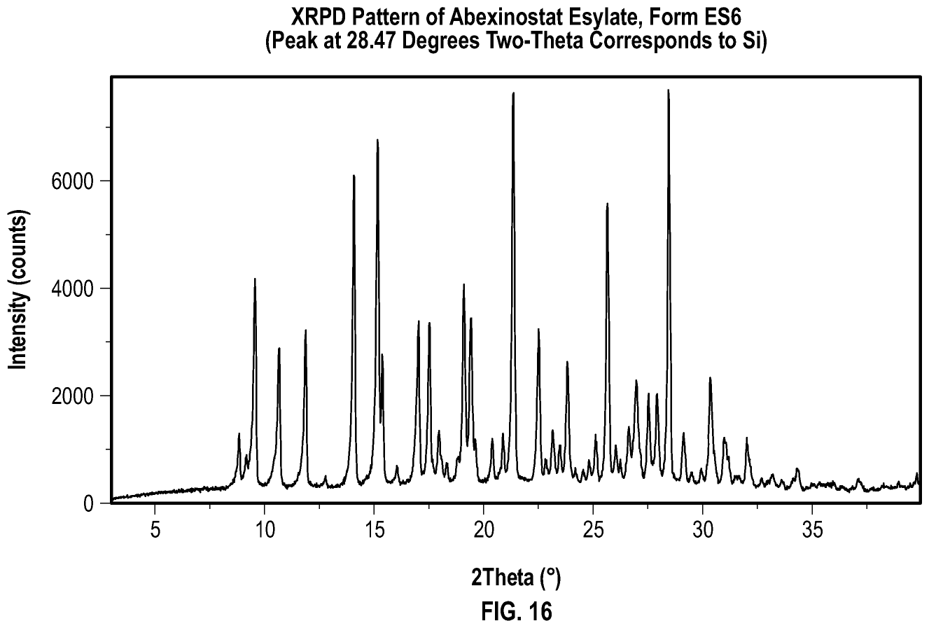

The present disclosure further includes a crystalline polymorph of Abexinostat esylate, designated Form ES6. The crystalline Form ES6 of Abexinostat esylate may be characterized by data selected from one or more of the following: an X-ray powder diffraction pattern substantially as depicted in FIG. 16; an X-ray powder diffraction pattern having peaks at 10.6, 11.9, 14.1, 23.8 and 25.7 degrees 2-theta±0.2 degrees 2-theta; and combinations of these data.

Crystalline Form ES6 of Abexinostat esylate may be further characterized by an X-ray powder diffraction pattern having peaks at 10.6, 11.9, 14.1, 23.8 and 25.7 degrees 2-theta±0.2 degrees 2-theta, and also having any one, two, three, four, or five additional peaks selected from 8.8, 16.0, 17.5, 20.9 and 23.5 degrees 2-theta±0.2 degrees 2-theta.

In one embodiment of the present disclosure, crystalline Form ES6 of Abexinostat esylate is isolated.

Crystalline Form ES6 of Abexinostat esylate may be an anhydrous form. Crystalline Form ES6 of Abexinostat esylate may be a hydrated form.

Crystalline Form ES6 of Abexinostat esylate may be characterized by each of the above characteristics alone/or by all possible combinations, e.g., an XRPD pattern having peaks at 10.6, 11.9, 14.1, 23.8 and 25.7 degrees 2-theta±0.2 degrees 2-theta; an XRPD pattern as depicted in FIG. 16, and combinations thereof.

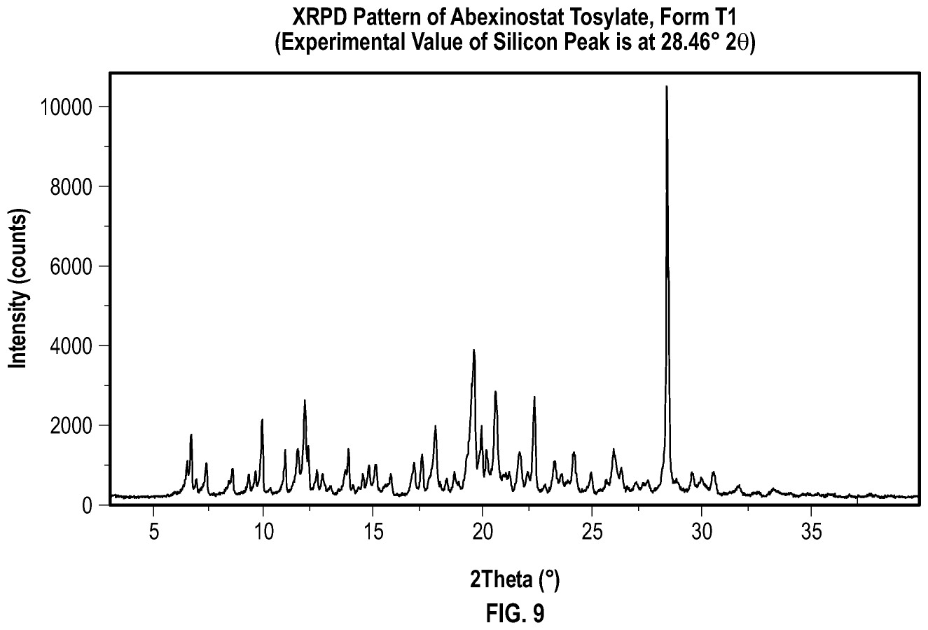

The present disclosure further includes a crystalline polymorph of Abexinostat tosylate, designated Form T1. Crystalline Form T1 of Abexinostat tosylate is a Abexinostat tosylate and p-toluenesulfonic acid monohydrate complex. The crystalline Form T1 of Abexinostat tosylate may be characterized by data selected from one or more of the following: an X-ray powder diffraction pattern substantially as depicted in FIG. 9; an X-ray powder diffraction pattern having peaks at 7.4, 8.6, 11.0, 12.1 and 22.4 degrees 2-theta±0.2 degrees 2-theta; and combinations of these data.

Crystalline Form T1 of Abexinostat tosylate may be further characterized by an X-ray powder diffraction pattern having peaks at 7.4, 8.6, 11.0, 12.1 and 22.4 degrees 2-theta±0.2 degrees 2-theta, and also having any one, two, three, four or five additional peaks selected from 16.9, 17.3, 20.6, 21.7 and 26.0 degrees 2-theta±0.2 degrees 2-theta.

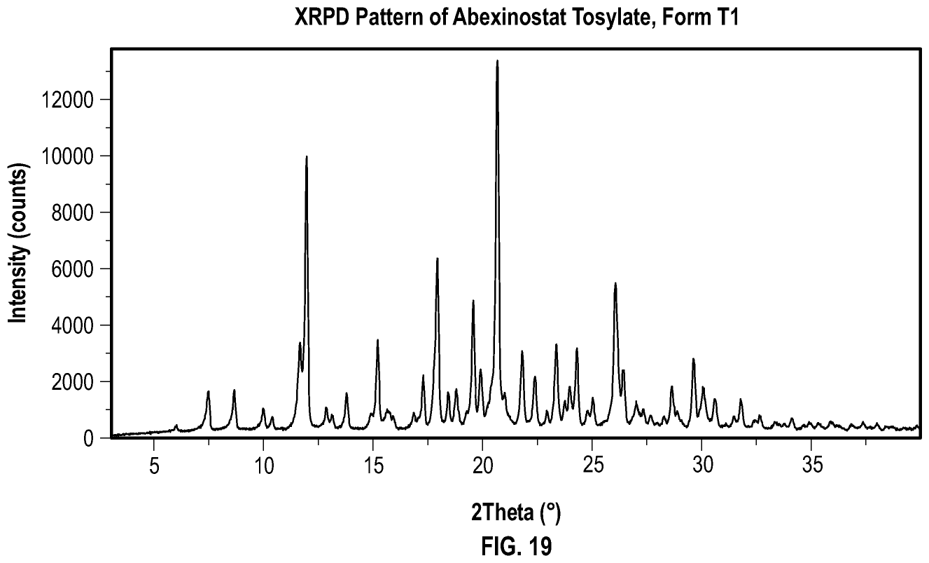

Crystalline Form T1 of Abexinostat tosylate may also be characterized by data selected from one or more of the following: an X-ray powder diffraction pattern substantially as depicted in FIG. 19; an X-ray powder diffraction pattern having peaks at 7.4, 8.6, 12.1 and 22.4 degrees 2-theta±0.2 degrees 2-theta; and combinations of these data.

Crystalline Form T1 of Abexinostat tosylate may be further characterized by an X-ray powder diffraction pattern having peaks at 7.4, 8.6, 12.1 and 22.4 degrees 2-theta±0.2 degrees 2-theta, and also having any one, two, three, four or five additional peaks selected from 16.9, 17.3, 20.6, 21.7 and 26.0 degrees 2-theta±0.2 degrees 2-theta.

Crystalline Form T1 of Abexinostat tosylate may also be characterized by an X-ray powder diffraction pattern having peaks at 11.9, 15.2, 20.7 and 23.3 degrees 2-theta±0.2 degrees 2-theta, and also having any one, two, three, four or five additional peaks selected from 11.6, 25.0, 28.6 and 29.6 degrees 2-theta±0.2 degrees 2-theta.

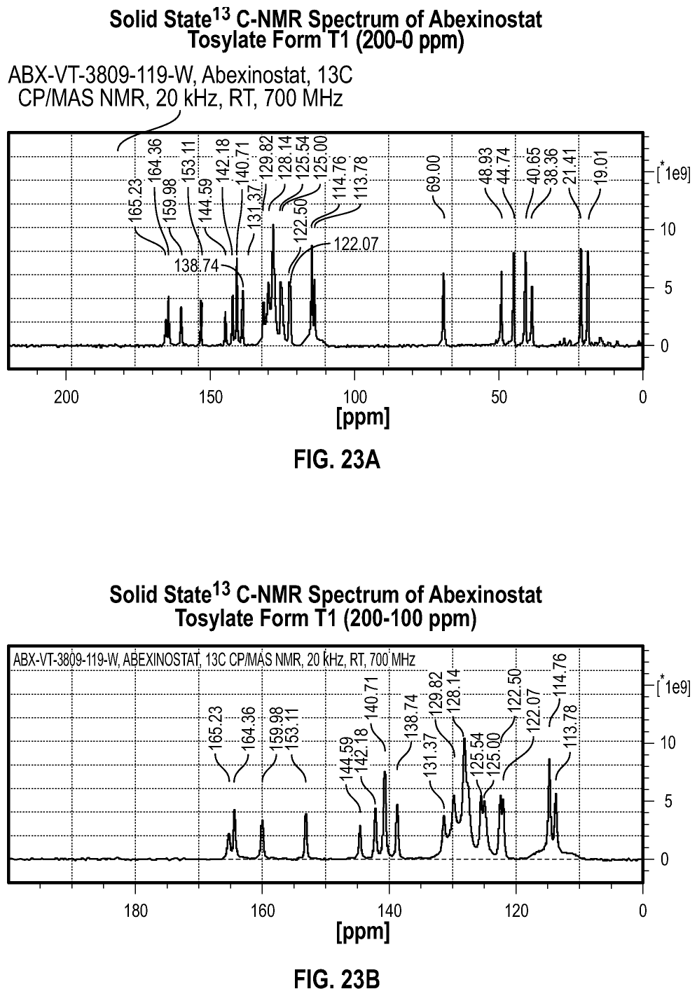

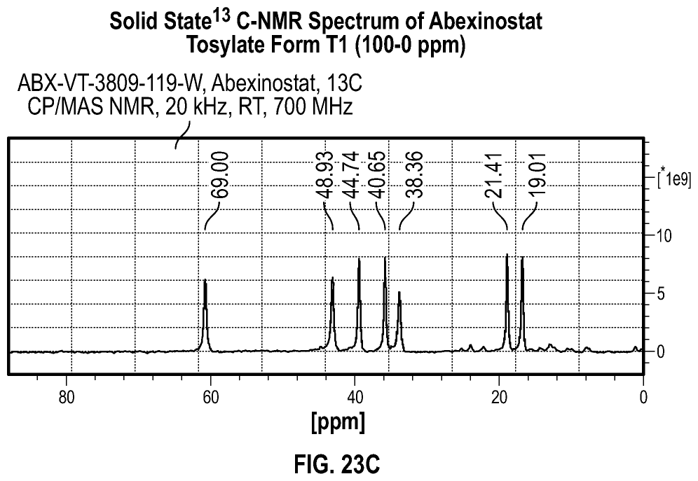

Crystalline Form T1 of Abexinostat tosylate may alternatively or additionally be characterized by data selected from one or more of the following: a solid state ¹³C NMR spectrum with characteristic peaks at 165.23, 164.36, 138.74, 69.00, 44.74, 40.65, and 38.36 ppm±0.2 ppm; or by a solid state ¹³C NMR having the following full peak list: 165.23, 164.36, 159.98, 144.59, 142.18, 140.71, 138.74, 131.37, 129.82, 128.14, 125.54, 125.00, 122.50, 114.76, 113.78, 69.00, 48.93, 44.74, 40.65, 38.36, 21.41, and 19.01 ppm±0.2 ppm; or by a solid state ¹³C-NMR spectrum substantially as depicted in FIGS. 23a, 23b and 23c; or combinations of these data.

In one embodiment of the present disclosure, crystalline Form T1 of Abexinostat tosylate is isolated.

Crystalline Form T1 of Abexinostat tosylate may be anhydrous form. Crystalline Form T1 may be a hydrate. Crystalline Form T1 of Abexinostat tosylate may be a co-crystal of Abexinostat tosylate with p-toluenesulfonic acid, specifically a co-crystal of Abexinostat tosylate with p-toluenesulfonic acid monohydrate. The ratio between Abexinostat tosylate to p-toluenesulfonic acid is 1:1.

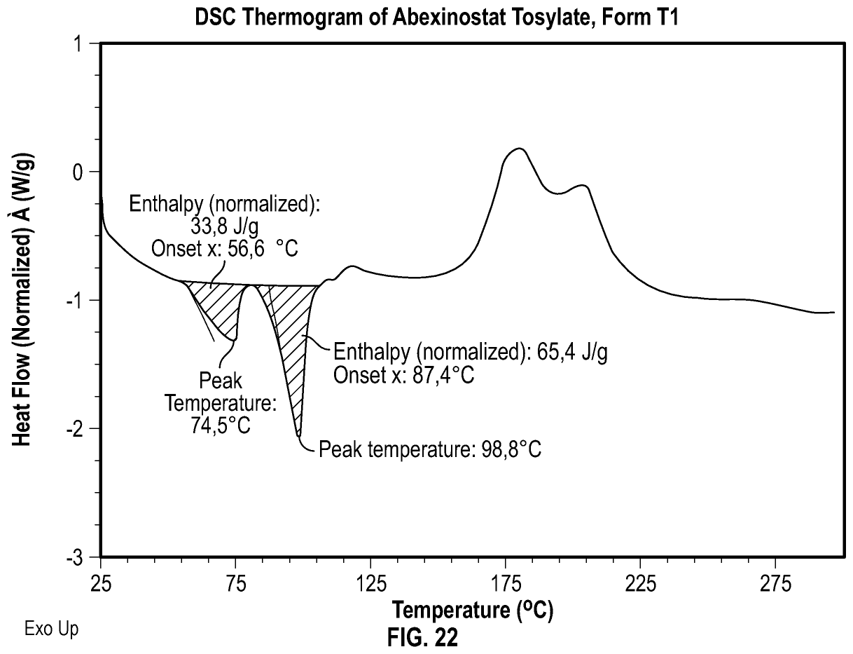

Crystalline Form T1 of Abexinostat tosylate may alternatively or additionally be characterized by a DSC thermogram as depicted in FIG. 22. Crystalline Form T1 of Abexinostat tosylate shows two endothermic peaks with an onset at 172° C.±3° C. and a melting point at around 176° C.

In one embodiment of the present disclosure, the space group and unit cell parameters of Form T1 of Abexinostat tosylate were determined using programs N-TREOR and DICVOL14 providing independently the identical result:

| Unit cell parameter | LeBail 293K |
|---|---|
| cell_length_a | 15.2203(8) Å |
| cell_length_b | 12.5257(7) Å |
| cell_length_c | 10.4317(7) Å |
| cell_angle_alpha | 104.031(7)° |
| cell_angle_beta | 96.000(7)° |
| cell_angle_gamma | 98.821(6)° |
| cell_volume | 1885.7(2) Å³ |
| symmetry_cell_setting | triclinic |
| Symmetry space group name | P-1 |

Crystalline Form T1 of Abexinostat tosylate is stable upon heating, pressure, grinding and upon exposure to humidity.

Crystalline Form T1 of Abexinostat tosylate may be characterized by each of the above characteristics alone/or by all possible combinations, e.g., an XRPD pattern having peaks at 7.4, 8.6, 11.0, 12.1 and 22.4 degrees 2-theta±0.2 degrees 2-theta; an XRPD pattern as depicted in FIG. 9, and combinations thereof, or e.g., an XRPD pattern having peaks at 7.4, 8.6, 12.1 and 22.4 degrees 2-theta±0.2 degrees 2-theta; an XRPD pattern as depicted in FIG. 19, and combinations thereof.

Crystalline Form T1 of Abexinostat tosylate can be prepared by
(1) suspending Abexinostat tosylate in a suitable solvent or solvent mixture;
(2) adding p-toluenesulfonic acid monohydrate;
(3) stirring the suspension until Abexinostat tosylate:p-toluenesulfonic acid monohydrate complex has formed; and
(4) optionally isolating crystalline Abexinostat tosylate:p-toluenesulfonic acid monohydrate complex.

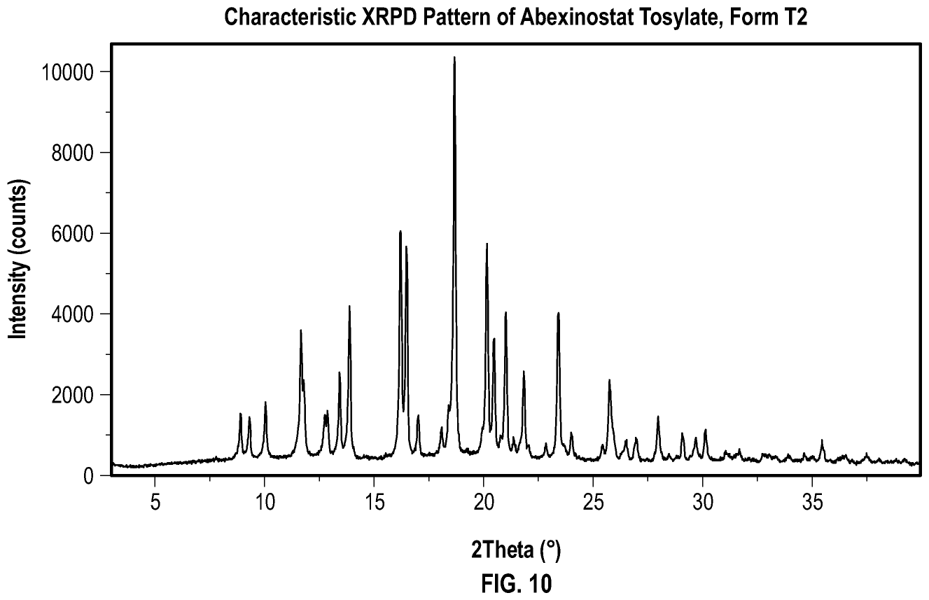

p-toluenesulfonic acid monohydrate should be added in an excess amount, preferably between 2-6 equivalents. Suitable solvents are ketone solvents, preferably methylisobutyl ketone (MIBK) or non-polar solvents, preferably toluene. Solvent mixtures can also be used. A suitable solvent mixture is MIBK/toluene, preferably the amount of toluene should be between 1-5% of toluene. The suspension can be stirred at a temperature between RT to 90° C., preferably between 30-80° C., most preferably between 40-70° C. The suspension may be stirred for a couple of days until Abexinostat tosylate:p-toluenesulfonic acid monohydrate complex has formed. Preferably, the suspension is stirred for 1 to 6 days at a temperature between 30-70° C. The present disclosure further includes a crystalline polymorph of Abexinostat tosylate, designated Form T2. The crystalline Form T2 of Abexinostat tosylate may be characterized by data selected from one or more of the following: an X-ray powder diffraction pattern substantially as depicted in FIG. 10; an X-ray powder diffraction pattern having peaks at 11.7, 16.2, 16.5, 20.2 and 21.9 degrees 2-theta±0.2 degrees 2-theta; and combinations of these data.

Crystalline Form T2 of Abexinostat tosylate may be further characterized by an X-ray powder diffraction pattern having peaks at 11.7, 16.2, 16.5, 20.2 and 21.9 degrees 2-theta±0.2 degrees 2-theta, and also having any one, two, three, four or five additional peaks selected from 8.9, 9.3, 13.4, 17.0 and 20.5 degrees 2-theta±0.2 degrees 2-theta.

In one embodiment of the present disclosure, crystalline Form T2 of Abexinostat tosylate is isolated.

Crystalline Form T2 of Abexinostat tosylate may be an anhydrous form. Crystalline Form T2 of Abexinostat tosylate may be a complex of Abexinostat tosylate with 1-butyl-3-methylimidazolium bis(trifluoromethanesulfonyl)imide.

Crystalline Form T2 of Abexinostat tosylate may be characterized by each of the above characteristics alone/or by all possible combinations, e.g., an XRPD pattern having peaks at 11.7, 16.2, 16.5, 20.2 and 21.9 degrees 2-theta±0.2 degrees 2-theta; an XRPD pattern as depicted in FIG. 10, and combinations thereof.

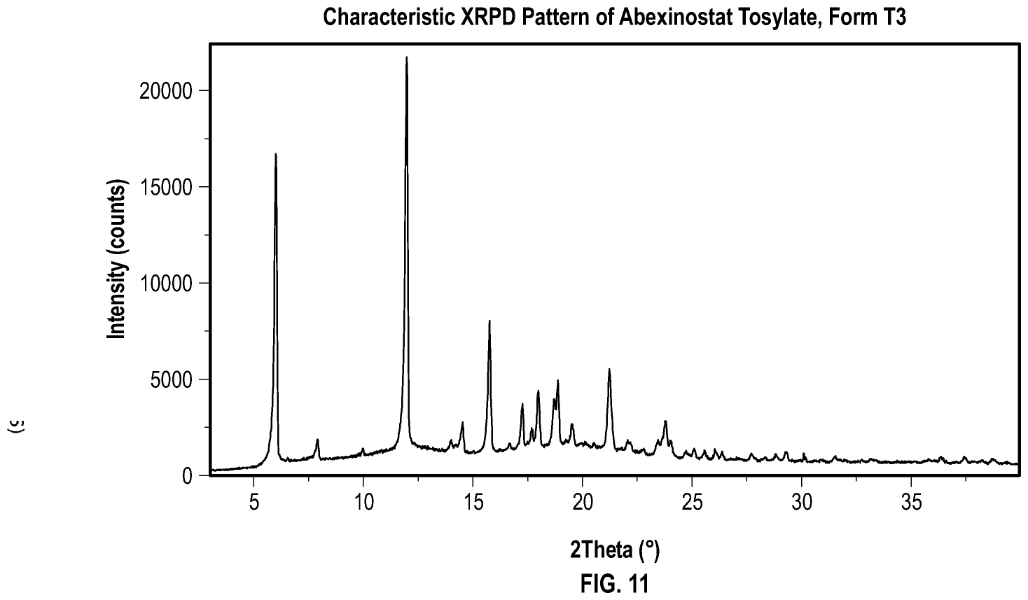

The present disclosure further includes a crystalline polymorph of Abexinostat tosylate, designated Form T3. The crystalline Form T3 of Abexinostat tosylate may be characterized by data selected from one or more of the following: an X-ray powder diffraction pattern substantially as depicted in FIG. 11; an X-ray powder diffraction pattern having peaks at 6.0, 12.0, 15.8, 17.3 and 21.2 degrees 2-theta±0.2 degrees 2-theta; and combinations of these data.

Crystalline Form T3 of Abexinostat tosylate may be further characterized by an X-ray powder diffraction pattern having peaks at 6.0, 12.0, 15.8, 17.3 and 21.2 degrees 2-theta±0.2 degrees 2-theta, and also having any one, two, three, four or five additional peaks selected from 7.9, 14.6, 17.7, 19.5 and 23.8 degrees 2-theta±0.2 degrees 2-theta.

In one embodiment of the present disclosure, crystalline Form T3 of Abexinostat tosylate is isolated.

Crystalline Form T3 of Abexinostat tosylate may be a hydrated form. Crystalline Form T3 of Abexinostat tosylate may be a complex of Abexinostat tosylate with 1-butyl-3-methylimidazolium bis(trifluoromethanesulfonyl)imide.

Crystalline Form T3 of Abexinostat tosylate may be characterized by each of the above characteristics alone/or by all possible combinations, e.g., an XRPD pattern having peaks at 6.0, 12.0, 15.8, 17.3 and 21.2 degrees 2-theta±0.2 degrees 2-theta; an XRPD pattern as depicted in FIG. 11, and combinations thereof.

The above crystalline polymorphs can be used to prepare other crystalline polymorphs of Abexinostat, Abexinostat salts and their solid state forms.

The present disclosure encompasses a process for preparing other solid state forms of Abexinostat, Abexinostat salts and their solid state forms. The process includes preparing any one of the Abexinostat salts and their solid state forms by the processes of the present disclosure, and converting that salt to another Abexinostat salt. The conversion can be done, for example, by a process including basifying any one or a combination of the above described Abexinostat salts and/or solid state forms thereof, and reacting the obtained Abexinostat base with an appropriate acid, to obtain the corresponding salt. Alternatively, the conversion can be done by salt switching, i.e., reacting a Abexinostat acid addition salt, with an acid having a pKa which is lower than the pKa of the acid of the first Abexinostat acid addition salt.

The present disclosure provides the above described crystalline polymorphs of Abexinostat and Abexinostat salts for use in the preparation of pharmaceutical compositions comprising Abexinostat or Abexinostat salts and/or crystalline polymorphs thereof.

The present disclosure also encompasses the use of crystalline polymorphs of Abexinostat of the present disclosure for the preparation of pharmaceutical compositions of crystalline polymorph Abexinostat and/or crystalline polymorphs thereof.

The present disclosure includes processes for preparing the above mentioned pharmaceutical compositions. The processes include combining any one or a combination of the crystalline polymorphs of Abexinostat of the present disclosure with at least one pharmaceutically acceptable excipient.

Pharmaceutical combinations or formulations of the present disclosure contain any one or a combination of the solid state forms of Abexinostat of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Abexinostat and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Abexinostat or Abexinostat salt can be administered. Abexinostat may be formulated for administration to a mammal, in embodiments to a human, by injection. Abexinostat can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Abexinostat and Abexinostat salts and the pharmaceutical compositions and/or formulations of Abexinostat and Abexinostat salts of the present disclosure can be used as medicaments, in embodiments in the treatment of cancer.

The present disclosure also provides methods of treating cancer by administering a therapeutically effective amount of any one or a combination of the crystalline polymorphs of Abexinostat or Abexinostat salts of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

Sample after being powdered in a mortar and pestle is applied directly on a silicon plate holder. The X-ray powder diffraction pattern was measured with Philips X'Pert PRO X-ray powder diffractometer, equipped with Cu irradiation source=1.54184 Å (Ångström), X'Celerator (2.022° 2θ) detector. Scanning parameters: angle range: 3-40 deg., step size 0.0167, time per step 37 s, continuous scan. The described peak positions were determined with or without using silicon powder as an internal standard in an admixture with the sample measured. The position of the silicon (Si) peak was corrected to silicone theoretical peak: 28.450 2θ, and the positions of the measured peaks were corrected respectively. In case such correction was applied, the Si-peak is indicated in the figures.

### Differential Scanning Calorimetry (DSC) Method

DSC measurements were done using TA Instruments Discovery, DSC unit. 1-3 mg of sample was weighted in pan, hermetically closed with the pin hole. Sample was purged with 50 ml/min of nitrogen flow and heated in the range of 25-350° C., with heating rate of 10° C./min.

Solid State NMR (ssNMR) Method

The ¹³C CP/MAS NMR spectra employing cross-polarization are acquired using the standard cross-polarization pulse scheme at spinning frequency of 15 kHz. The cross-polarization contact time is usually 2 ms, and the dipolar decoupling SPINAL64 is applied during the data acquisition. The number of scans is set for the signal-to-noise ratio SINO reaches at least the value ca. 50. The ¹³C scale is referenced to α-glycine (176.03 ppm for ¹³C). The ¹H MAS NMR spectra are measured at MAS frequency of 15 kHz using a single-pulse experiment with the number of scans 32.

For the correct measurements of all the above-mentioned spectra, the recycle delay D1 is optimized experimentally by the measurement of ¹H MAS NMR spectra with variable repetition delay. In this optimization the ¹H MAS NMR spectra are recorded with dummy scans, the number of which is DS=8 and number of scans NS=1. The D1 delay is gradually (step-by-step) increased from the initial value D1=0.5 s by multiplying using the factor n=2. This way, the build-up of intensity of ¹H NMR signals is monitored. The whole procedure is finished when the constant (equilibrium) signal intensity is reached. The repetition delay D1 used for the measurement of CP/MAS NMR spectra is then set to be 80% of the obtained equilibrium value.

### EXAMPLES

### Preparation of Starting Materials

Abexinostat base can be prepared according to methods known from the literature, for example International Publication No. WO 2004/092115. Abexinostat tosylate, Form I can be prepared according to methods known from the literature, for example International Publication No. WO 2014/0135776.

### Example 1: Preparation of Abexinostat Form A

24 mg of Abexinostat base was dissolved in 0.2 mL of acetic acid at room temperature. Solution was added dropwise in 4 mL of acetone. The obtained suspension was stirred for about 15 minutes and vacuum filtered. The sample was analyzed by XRPD and Abexinostat base Form A was obtained.

### Example 2: Preparation of Abexinostat HCl, Form H1

Abexinostat base (845 mg) was suspended in 20 ml of 2-propanol at room temperature. Hydrochloric acid (eq. 155 mg) was added into suspension. The suspension was heated to 75° C. and 2.5 mL of water was added to achieve complete dissolution. The solution was then put into an ice bath. After crystallization occurred, crystals were filtrated off and analyzed by XRPD. Abexinostat hydrochloride, Form H1 (dihydrate) was obtained.

### Example 3: Preparation of Abexinostat HCl, Form H2

Abexinostat hydrochloride, form H1 (1 mg) was placed in a pin hole aluminum pan. The sample was subjected to thermal treatment in DSC Discovery TA instruments, according to following steps:
1. Heating of the sample by heating rate 10° C./minute up to temperature of 120° C.;
2. Isothermal heating for 30 minutes at 120° C.;
3. Cooled to room temperature; and
4. Heating of the sample by heating rate 10° C./minute up to temperature of 300° C.

The obtained solid was analyzed by XRPD. Abexinostat hydrochloride, Form H2 (anhydrous) was obtained.

### Example 4: Preparation of Abexinostat Fumarate, Form F1

Abexinostat base (154.8 mg) was suspended in 2 ml of water at room temperature. Fumaric acid (eq. 45.2 mg) was added into suspension. The suspension was dissolved at 40° C. and the solution was put into an ice bath. After crystallization occurred, crystals were filtrated off and analyzed by XRPD. Abexinostat fumarate, form F1 was obtained.

### Example 5: Preparation of Abexinostat Maleate, Form M1

Abexinostat base (946.9 mg) was suspended in 30 ml of methyl-ethylketone (MEK) at room temperature. Maleic acid (eq. 553.1 mg) was added into the suspension. The suspension was then heated to 40° C. and the obtained solution was put into an ice bath. After crystallization occurred, crystals were filtrated off and analyzed by XRPD. Abexinostat maleate, Form M1 (anhydrous) was obtained.

### Example 6: Preparation of Abexinostat Fumarate, Form F2

Abexinostat base (1 eq., 1893.8 mg) was suspended in 60 ml of water at 20-25° C. Fumaric acid (2 eq., 1106.2 mg) was added into suspension. The suspension was dissolved at 44° C. and obtained solution was cooled to 0-5° C. After crystallization occurred, crystals were filtrated off and analyzed by XRPD. Abexinostat fumarate, Form F2 was obtained.

### Example 7: Preparation of Abexinostat Mesylate, Form MS1

Abexinostat base (1 eq., 337 mg) was suspended in 5 ml of acetonitrile at 20-25° C. Methanesulfonic acid (2 eq., 110.1 mg) was added into suspension after which solution was obtained. Crystallization occurred at 20-25° C. and suspension was heated to 70° C., then it was again cooled to RT. Crystals were filtrated off and analyzed by XRPD. Abexinostat mesylate, Form MS1 was obtained.

### Example 8: Preparation of Abexinostat Edisylate, Form E1

Abexinostat base (1 eq., 233.8 mg) and 1,2-Ethanedisulfonic acid dihydrate (2 eq., 266.2 mg) was suspended in 5 ml of acetone at 20-25° C. Solution was obtained after several minutes. Crystallization occurred overnight at 20-25° C. Crystals were filtered off and analyzed by XRPD. Abexinostat edisylate form E1 was obtained.

### Example 9: Preparation of Abexinostat Tosylate, Form T1

Abexinostat tosylate, form I (120 mg) and p-toluene sulfonic acid monohydrate (2 eq., 80 mg) were suspended in 3 ml of MIBK and stirred for 3 days at 60° C. The suspension was then vacuum filtered and vacuum dried at 70° C. for 2 hours. Sample was analyzed by XRPD, Abexinostat tosylate, Form T1 was obtained.

### Example 10: Preparation of Abexinostat Tosylate, Form T2

Abexinostat tosylate, Form I (200 mg) was dissolved in 2.5 ml of solvent mixture methanol: 1-butyl-3-methylimidazolium bis(trifluoromethanesulfonyl)imide (5% of ionic liquid) at 54° C. Solution was slowly cooled to room temperature but crystallization did not occur. 10 ml of water was added dropwise into the solution and crystallization occurred. The suspension was stirred for 3 additional hours and vacuum filtered. Sample was analyzed by XRPD and Abexinostat tosylate, Form T2 was obtained.

### Example 11: Preparation of Abexinostat Tosylate, Form T3

Abexinostat tosylate, Form I (500 mg) was dissolved in 6.3 ml of solvent mixture methanol: 1-butyl-3-methylimidazolium bis(trifluoromethanesulfonyl)imide (10% of ionic liquid) at 54° C. Solution was slow cooled to room temperature but crystallization did not occur. 25 ml of water was added dropwise into the solution and crystallization occurred momentarily. Suspension was stirred for 5 additional hours and vacuum filtered. Sample was analyzed by XRPD and Abexinostat tosylate, Form T3 was obtained.

### Example 12: Preparation of Abexinostat Fumarate, Form F3

Abexinostat base (151.7 mg) was suspended in 2 ml of acetone at room temperature. Suspension was heated to 40° C. Fumaric acid (eq. 48.6 mg) was added into the suspension. The slurry was stirred at 40° C. for 5 hours and then cooled down to room temperature. The slurry was filtrated off and analyzed by XRPD. Abexinostat fumarate, Form F3 was obtained.

### Example 13. Preparation of Abexinostat Edisylate, Form E2

Abexinostat base (732.5 mg, 1.843 mmol) and 1,2-Ethanedisulfonic acid monohydrate (767.5 mg, 3.686 mmol) were suspended in 10.0 ml of EtOH, abs. at room temperature. The obtained suspension was heated to reflux at about 70° C. and stirred for 2 hours, then cooled down to room temperature and left to stir overnight at room temperature. The slurry was filtrated off with vacuum, dried in a vacuum oven at 50° C. for 3 hours and analyzed by XRPD. Abexinostat edisylate, Form E2 was obtained.

### Example 14. Preparation of Abexinostat Esylate, Form ES1

Abexinostat base (643.4 mg, 1.619 mmol) was suspended in 9.0 ml of acetone at room temperature. Ethanesulfonic acid was quickly added into the suspension (266.1 μl, 3.238 mmol). The obtained suspension was heated to reflux at about 50° C. and stirred for 2 hours, then cooled down to room temperature and left to stir overnight at room temperature. The slurry was filtrated off with vacuum and analyzed by XRPD. Abexinostat esylate, Form ES1 was obtained.

### Example 15. Preparation of Abexinostat Esylate, Form ES5

Abexinostat base (643.4 mg, 1.619 mmol) was suspended in 6.5 ml of acetonitrile at room temperature. Ethanesulfonic acid was quickly added into the suspension (266.1 μl, 3.238 mmol). The obtained suspension was heated to reflux at about 50° C. and stirred for 2 hours, then cooled down to room temperature and left to stir overnight at room temperature. The slurry was filtrated off with vacuum, dried in a vacuum oven at 50° C. for 3 hours and analyzed by XRPD. Abexinostat esylate, Form ES5 was obtained.

### Example 16. Preparation of Abexinostat Esylate, Form ES6

Abexinostat base (643.4 mg, 1.619 mmol) was suspended in 9.0 ml of acetone at room temperature. Ethanesulfonic acid was slowly added dropwise into suspension (266.1 μl, 3.238 mmol). The obtained suspension was heated to reflux at about 50° C. and stirred for 2 hours, then cooled down to room temperature and left to stir overnight at room temperature. The slurry was filtrated off with vacuum and analyzed by XRPD. Abexinostat esylate, Form ES6 was obtained.

### Example 17. Preparation of Abexinostat Edisylate, Form E6

Abexinostat base (732.5 mg, 1.843 mmol) and 1,2-Ethanedisulfonic acid monohydrate (767.5 mg, 3.686 mmol) were suspended in 12.0 ml of EtOH, abs. at room temperature. The obtained suspension was heated to reflux at about 70° C. and stirred for 2 hours, then cooled down to room temperature and left to stir overnight at room temperature. The slurry was filtrated off with vacuum, dried in a vacuum oven at 60° C. for 3 hours and analyzed by XRPD. Abexinostat edisylate, Form E6 was obtained.

### Example 18. Preparation of Abexinostat Edisylate, Form E7

Abexinostat base (732.5 mg, 1.843 mmol) and 1,2-Ethanedisulfonic acid monohydrate (767.5 mg, 3.686 mmol) were suspended in 12.0 ml of EtOH (dried at molecular seeds) at room temperature. The obtained suspension was heated to reflux at about 70° C. and stirred for 2 hours, then cooled down to room temperature and left to stir overnight at room temperature. The slurry was filtrated off with vacuum, dried in a vacuum oven at 60° C. for 3 hours and analyzed by XRPD. Abexinostat edisylate, Form E7 was obtained.

### Example 19. Preparation of Abexinostat Tosylate and p-Toluenesulfonic Acid Complex, Form T1

50 mg of Abexinostat tosylate and 50 mg of p-toluenesulfonic acid monohydrate (3 eq.) were suspended in 1 ml of solvent mixture MIBK:toluene (2% of toluene) and stirred for 4 days at 40° C. The suspension was vacuum filtered after 4 days and analyzed by XRPD. Abexinostat tosylate and p-toluenesulfonic acid monohydrate complex, Form T1 was obtained (FIG. 19).

### Example 20. Preparation of Abexinostat Maleate Form M1

30 mg of Abexinostat maleate was dissolved in 1.5 mL of 1-propanol at 97° C. The solution was left at room temperature without stirring for 1 day after which crystallization occurred. The precipitate was filtered and analyzed by XRPD. Abexinostat maleate Form M1 was obtained.

### Example 21. Preparation of Abexinostat Fumarate Form F3

20 mg of Abexinostat fumarate was dissolved in 0.5 mL of acetone/water mixture (1:1) at room temperature. The solution was left at room temperature without stirring for 10 days after which crystallization occurred. The precipitate was filtered and analyzed by XRPD. Abexinostat fumarate Form F3 was obtained.

1. Crystalline Abexinostat tosylate:p-toluenesulfonic acid.

2. Crystalline Abexinostat tosylate:p-toluenesulfonic acid monohydrate.

3. Crystalline Abexinostat tosylate:p-toluenesulfonic acid monohydrate, which is a co crystal.

4. A crystalline product according to claim 3, designated Form T1, which is characterized by data selected from one or more of the following:
a) an XRPD pattern having peaks at 7.4, 8.6, 11.0, 12.1 and 22.4 degrees 2-theta 0.2 degrees 2-theta;
b) an XRPD pattern as depicted in FIG. 9;
c) an XRPD pattern having peaks at 7.4, 8.6, 12.1 and 22.4 degrees 2-theta±0.2 degrees 2-theta;
d) an XRPD pattern as depicted in FIG. 19;
e) an 13C ssNMR spectrum having peaks at 165.23, 164.36, 138.74, 69.00, 44.74, 40.65, and 38.36 ppm±0.2 ppm;
f) an 13C ssNMR spectrum as depicted in FIG. 23;
g) unit cell data: cell_length_a 15.2203 A cell_length, b 12.5257 Å, cell_length_c 10.4317 A, cell angle alpha 104.032°, cell_angle_beta 96.000°, cell angle gamma 98.821°, Symmetry space group name PI; and
h) combinations of these data.

5. A crystalline product according to claim 4, designated Form T1, characterized by the XRPD pattern having peaks at 7.4, 8.6, 12.1 and 22.4 degrees 2-theta±0.2 degrees 2-theta, and also having one, two, three or four additional peaks selected from 16.9, 17.3, 20.6, 21.7 and 26.0 degrees two theta±0.2 degrees two theta.

6. A process for the preparation of a crystalline product according to claim 3 designated Form T1, comprising:
1) suspending Abexinostat tosylate in a suitable solvent or solvent mixture;
2) adding p-toluenesulfonic acid monohydrate;
3) stirring the suspension until Abexinostat tosylate:p-toluenesulfonic acid monohydrate complex has formed; and
4) optionally isolating crystalline Abexinostat tosylate:p-toluenesulfonic acid monohydrate complex.

7. A process according to claim 6, further comprising combining the crystalline product with at least one pharmaceutically acceptable excipient to provide a pharmaceutical composition.

8. A crystalline product obtainable by a process according to claim 6.

9. A pharmaceutical composition comprising a crystalline product produced according to claim 6 and at least one pharmaceutically acceptable excipient.

10. (canceled)

11. A process comprising combining a crystalline product according to claim 3 with at least one pharmaceutically acceptable excipient.

12. A medicament comprising the crystalline product according to claim 3.

13. (canceled)

14. A method of treating cancer, comprising administering a therapeutically effective amount of a crystalline product according to claim 3 to a subject in need of the treatment.

15-26. (canceled)
